(19) United States
(12) Patent Application Publication
(10) Pub. No.: US 2003/0175804 A1
(43) Pub. Date: Sep. 18, 2003

(54) Macromolecule detection

(76) Inventor: Peter James, Lund (SE)

Correspondence Address: BOZICEVIC, FIELD & FRANCIS LLP, 200 MIDDLEFIELD RD, SUITE 200, MENLO PARK, CA, 94025, US

(21) Appl. No.: 10/240,700

(22) PCT Filed: Apr. 3, 2001
(86) PCT No.: PCT/AU01/00366

(30) Foreign Application Priority Data
Apr. 3, 2000 (AU) PQ6643

(51) Int. Cl. G01N 33/53; C12P 21/06

(52) U.S. Cl. 435/7.1; 435/68.1

(57) ABSTRACT

A method is provided of labelling a protein, which method comprises the steps of (a) treating the protein with a protecting agent so as to block epsilon amino acid groups present on lysines residues of the protein, (b) cleaving the protein into a peptide mixture; and (c) treating the peptide mixture with a labelled agent which binds to N-terminal amino acids of the peptides. The method may be used in differential expression detection methods for determining differences in protein expression.

### TECHNICAL FIELD

The present invention relates to methods of quantitating, identifying and characterising proteins and peptide fragments.

### BACKGROUND TO THE INVENTION

Despite its limitations, the best established and most widely applicable approach to study protein expression quantitatively is by densitometric analysis of protein extracts separated by two-dimensional polyacrylamide gel electrophoresis (2D-PAGE). Nevertheless, this method is not always reliable due to protein specific differences in staining intensity, the low reproducibility of the staining procedure, as well as problems due to proteins overlapping and streaking. Stable and radio-isotope in vivo metabolic labelling has provided a partial answer to this problem (1,2).

The problem of protein identification has been revolutionised by the introduction of methods to identify proteins in databases using mass spectral data, either based on peptide masses from protein digests (3), or fragmentation spectra from individual peptides (4,5). de novo protein sequencing and its automation has been the subject of intense interest since native peptide fragmentation (MS/MS) became possible (6,7). Isotopic labelling and fragmentation-directing charged derivatives have been investigated (8-10) as a means of simplifying spectral interpretation. However, no universal answer has been forthcoming and even the use of raw MS/MS spectra to search databases has its limitations. The need to deal with post-translational modifications and the ability to work with organisms whose genome has not been sequenced requires the development of new methods.

### SUMMARY OF THE INVENTION

The present inventors have developed a method for labelling proteins that allows relative protein quantitation in 1 and 2D gel separations even if the separation is only partial and facilitates de novo sequencing and automated interpretation of MS/MS fragmentation spectra. The method involves labelling mixtures of peptide fragments, obtained by cleaving a protein, with a labelling agent which binds to the N-terminal amino acid of the peptide fragments, wherein the the protein is treated prior to cleavage with a protecting agent so as to block epsilon amino acid groups present on lysines. Blocking of the epsilon amino acid groups is required to prevent the labelling agent from labelling internal lysines.

Accordingly, in a first aspect the present invention provides a method of labelling a protein, the method comprising the steps of:

(a) treating the protein with a protecting agent so as to block epsilon amino acid groups present on lysines;

(b) cleaving the protein into a peptide mixture; and

(c) treating the peptide mixture with an labelled agent which binds to N-terminal amino acids of the peptides.

Preferably, step (a) is succinylation, preferably using the protecting agent succinic anhydride.

Preferably, cleaving in step (b) is by incubation with Asp/GluC (V8) protease.

Preferably step (c) is treating with 1-(H4/D4 nicotinoyloxy)succinimide (H4 or D4 Nic-NHS) ester.

In a second aspect, the present invention provides a method of identifying and/or characterising a protein which method comprises labelling a protein by the method described in the first aspect followed the step of:

(d) detecting or measuring the amount of label on the peptides.

The method may further include the step of:

(e) identifying the amino acid sequence of the labelled peptides.

Preferably, step (e) is by mass spectral analysis using an ion trap mass spectrometer.

Preferably, the protein is separated from other proteins prior to labelling. Proteins of interest may, for example, be excised from 1D or 2D PAGE gels and analysed.

In a third aspect, the present invention provides a method of comparing or determining the expression of a protein in a first cell and a second cell, the method comprising the steps of:

(i) obtaining the protein from a first cell and a second cell;

(ii) labelling the protein from the first cell and the second cell by the method of the first aspect of the invention wherein a first labelling agent is used to label the the protein from the first cell and a second labelling agent is used to label the the protein from the second cell and wherein the first and second labelling agent can be distinguished; and

(iii) detecting or measuring the amount of first and second labelling agent on the peptides.

Preferably the first and second labelling agent can be distinguished on the basis of mass.

Preferably step (iii) is MALDI MS.

In a highly preferred embodiment, the first labelling agent comprises a light isotopic label and the second labelling agent comprises a heavy isotopic label, or vice versa. Preferably, the first and second labelling agents are 1-(H4 nicotinoyloxy) succinimide (H4 Nic-NHS) ester and 1-(D4 nicotinoyloxy) succinimide (D4 Nic-NHS) ester.

The above method may which further comprise the step of:

(iv) identifying the amino acid sequence of the labelled peptides.

Preferably step (iv) is by mass spectral analysis using an ion trap mass spectrometer.

In a specific embodiment of the third aspect, the present invention consists in a method of determining the expression of a protein in a cell under different states, the method comprising the steps of:

(a) obtaining the protein from a first and second expression state,

(b) treating the protein from the first and second states with a protecting agent so as to block epsilon amino acid groups present on lysines;

(c) cleaving the protein from the first and second states into peptide mixtures;

(d) treating the peptide mixture from the first state with a light isotopically-labelled agent which binds to N-terminal amino acids of the peptides;

(e) treating the peptide mixture from the second state with a heavy isotopically-labelled agent which binds to N-terminal amino acids of the peptides; and

(f) combining the treated peptide mixtures from the first and second states; and

(g) detecting for or measuring the amount of light and heavy label on the peptides.

In a fourth aspect, the present invention provides the use of two or more differentially isotopically-labelled succinylating agents as labelling agents for peptides and proteins. Preferably said agents are 1-(H4 or D4 nicotinoyloxy) succinimide (H4/D4 Nic-NHS) esters.

### DETAILED DESCRIPTION OF THE INVENTION

In the methods of the present invention, proteins, which have treated with cleaving agents to form a mixture of peptides are then treated with a labelling agent which labels the N-terminal amino acid of the peptides. Since the labelling agent reacts with the N-terminus by virtue of its reaction with free amino groups, it is necessary to pre-treat the proteins to block binding of the labelling agent to amino groups present on internal amino acid residues, specifically lysine. If the epsilon amino groups on lysine were not blocked, then the labelled agent would also bind to all free amino groups on the lysines making it difficult to interpret the amino acid sequence of the labelled peptide.

Accordingly, the protecting agent may be any suitable protecting agent which blocks the epsilon amino groups of lysine residues. A preferred agent is a succinylating agent such as succinic anhydride.

Once the protein has been treated with the protecting agent, then the protein is cleaved into fragments. This may be achieved by enzymatic or chemical means. Examples of suitable enzyme treatments include the use of proteases such as includes trypsin and other suitable proteases to generate peptides. Techniques for cleaving proteins into suitable fragments are kownin the art.

The next step is to treat the peptide mixture with a labelled agent which binds to the N-terminal amino acid of the peptides. The labelled agent comprises a detectable label. Preferably, the detectable label is one which can be detected by a method of mass determination such as mass spectroscopy.

More specifically, it is preferred to use labels that can be produced in two or more forms which confer the ability to distinguish the different forms of labelled agents and the peptides to which they are linked by mass, but which, importantly, do not affect the ionisation efficiency of the peptides to which they are linked when subjected to mass spectroscopy.

A particularly preferred detectable label is an atom which can be incorporated into the labelled agent in different isotopic forms, for example hydrogen and its heavier forms deuterium and tritium. Other examples include carbon-12 and its heavier form carbon-14.

The labelling agent may be any moiety which reacts with an N-terminal amino group. In a preferred embodiment, the labelling agent is a 1-nicotinoyloxy succinimide ester, more preferably 1-(H4/D4-nicotinoyloxy) succinimide ester.

The protein labelling methods of the invention maybe used to label proteins for subsequent detection steps which typically comprise detecting, quantitatively, the presence of the detectable label. Examples of suitable detection methods include MALDI mass spectroscopy.

Detection methods may further comprise techniques for identifying the amino acid sequence of the labelled peptides. Suitable techniques include mass spectral analysis using an ion trap spectrometer.

The use of two or more labelling agents with different labels, allows a determination of relative amounts of proteins in two or more different samples. In particular, the labelling techniques of the present invention may be used to compare protein expression in two different cells. The two different cells may, for example, be cells of the same type but under different conditions (or states), or they may be cells of a different type (under the same or different conditions). Thus, by way of example, a first cell may be treated with an agonist and a second cell untreated, and the expression of one or more proteins in each cell compared.

The two conditions could also be cells resting versus cells induced or treated in some manner. Often, differential expression in cells under different conditions can provide useful information on the activity in the cells.

The comparison is achieved according to the present invention by labelling the protein or proteins from the first cell by the methods of the invention using a first labelling agent and the protein or proteins from the second cell by the methods of the invention using a second labelling agent. The first and second labelling agents comprise a different detectable label such that they can be distinguished. Preferably they may be distinguished by virtue of differing masses. As discussed above, when the peptides are to be detected by mass spectroscopy, it is important that the differences between the different labels do not substantially affect the ionisation characteristics of the peptides to which they are linked. Thus the different labels should preferably be chosen such that the ionisation products of any given peptide to which they linked are substantially identical, except for a consistent different in mass due to the difference in mass of the different labels. Specific examples of suitable labels and labelling agents, as discussed above, are labels and agents that differ by virtue of comprising different isotopes of a particular atom, such as hydrogen or carbon. In other words preferred labelled agents are those where one labelled agent comprises a heavy atom label (a heavy label) and one labelled agent comprises a light atom label (a light label).

The protein or proteins from the first and second cell are typically separated from other proteins by techniques such as chromatography or gel electrophoresis and then labelled by the methods of the present invention. For example, whole cell lysates may be separated by 1D or 2D gel electrophoresis and equivalent bands excised.

The mixtures of labelled peptides obtained from the first and second cells after treatment of the proteins may then be measured separately or combined and then measured together. If measured separately, steps should be taken to standardise results such that a quantitative comparison can be performed.

If equivalent amounts of total protein form both first and second cells are labelled and measured, useful information can be obtained using the present invention on the amount and type of expression of a protein under different conditions. For example, where a light agent and a heavy label are used, if the ratio of light to heavy label on the peptides is the same, then it can be inferred that there was no change in expression of that protein in the second state. If the ratio of heavy to light increases, then the expression of the protein has increased. If no heavy label is detected, then either the expression of the protein was stopped in the second state or some change in the protein has occurred resulting in movement to some other location on the gel.

The method for quantitation and de novo sequencing according to the present invention is universally applicable and is does not require a protein to contain a certain amino acid such as cysteine or lysine. Moreover, the method does not require in vivo metabolic labelling. The method is ideally suited for the analysis of changes in membrane protein expression since these proteins can be partially separated by 1D SDS-PAGE and the labelling allows one to quantitate multiple proteins in a single band. The method is also open to automation and allows flexible sequence searching in databases, allowing for differences due to homologies and post-translational modifications.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In order that the present invention may be more clearly understood preferred forms will be described with reference to the following examples and the accompanying drawings.

### BRIEF DESCRIPTION OF DRAWINGS

FIG. 1. Protein quantitation and sequencing strategy. Extracts from cells obtained under different conditions are separated by two-dimensional SDS-PAGE electrophoresis. Individual spots are selected and excised and the lysine residues are succinylated before digestion with Asp/GluC protease. Peptides obtained from state 1 are specifically labelled at the N-terminal with the light reagent H4NicNHS (1-(H4-nicotinoyloxy) succinimide) and the corresponding ones from state 2 with the heavy reagent D4NicNHS. The digests are combined and a fraction is analysed by MALDI MS in order to quantitate the relative amount of the proteins from the D4/H4 ratios of the individual peptides. Those peptides coming from proteins showing a change in expression levels are then analysed by tandem mass spectrometry.

FIG. 2. Peptide sequencing by MS/MS. An Escherichia coli protein was succinylated and then digested with a mixture of V8-protease and trypsin. The digest was H4/D4-nicotinylated and desalted on a microtip containing C18 beads and then analysed by nano-electrospray ionisation in an ion trap mass spectrometer. The singly charged parent ion isotope cluster 952/956 clearly shows the characteristic isotopic distribution due to the modification (zoom scan shown in insert in A). The peptide cluster was isolated and subjected to CAD MS/MS (B). All the b ions appear as doublets separated by four mass units whilst the y ions are just singlets. The sequence can be rapidly read out from the mass differences between adjacent doublets. Due to the limited mass range of the ion trap under MS/MS conditions only a partial sequence was read. The full sequence can be obtained by MS/MS/MS analysis of the smallest doublet in the spectrum (422/426, b3). The complete sequence of the peptide was deduced (NMAGSLVR) and the protein identified by FASTA searching as the 50S ribosomal subunit protein L17.

FIG. 3. Two dimensional SDS-PAGE separations of E. coli grown in full (A) and carbon limited medium (B). The positions of the proteins excised from the two gels are shown in panel A

FIG. 4. MALDI mass spectrum of the combined digests of spots 37 shown in FIG. 3. The spectrum shows that two species are present: one series of peptides labelled with an asterisk arise from a protein identified as recA (DNA-dependent ATPase) by MS/MS sequencing whose expression level is not changing. The second series arises from a protein undergoing a three fold increase under carbon limitation conditions which was identified as malE (periplasmic maltose-binding protein).

### MODES FOR CARRYING OUT THE INVENTION

### Experimental Protocol

Synthesis of the 1-(H4/D4 nicotinoyloxy) succinimide (H4/D4-Nic-NHS) esters

Nicotinic acid was dissolved in dry tetrahydrofuran and mixed with one equivalent of dicyclohexylcarbodiimide under continuous stirring in a reaction flask for 2 hours at room temperature. One equivalent of N-hydroxysuccinimide were added to the solution and stirred over night at room temperature. The precipitate was recovered by filtration and purified by recrystallisation from ethyl acetate.

Chemical Modification of Proteins

Escherichia coli MC4100 was obtained from the laboratory collection (16) and the bacteria were cultivated in a synthetic medium with either 5 or 100 mM Glucose as the sole carbon source. Sample preparation and 2D-gel analysis was carried out as described previously (17). Gels were scanned in a Personal Laser Densitometer (Molecular Dynamics, Sunnyvale, Calif., USA) and image analysis, spot matching and quantification were performed using the 2D™ software package (PDQuest, Pharmacia, Uppsala, Sweden) on a PowerMac. Either the entire spot or a 1 mm² diameter circular gel piece was cut from the centre of the spots chosen for analysis and completely destained in ethanol containing 0.5% v/v trimethylamine. The spot was washed in water then dehydrated in acetonitrile. 100 mM succinic anhydride was freshly prepared in 2M Urea and 200 mM sodium phosphate buffer and the pH rapidly adjusted to 8.5 with sodium hydroxide. The spot was rehydrated in 100 μL of the reagent solution and succinylation was allowed to proceed for 2 hours before a fresh solution of reagent was added. The gel spot was desalted by four alternate additions of pure water or acetonitrile. After the last acetonitrile wash, the spot was rehydrated with 10 μl of 2M urea in 20 mM pH 7.8 sodium phosphate buffer containing 1 μg Asp/GluC (V8) protease and digestion was carried out at 37° C. for six hours. The peptide mixture was then N-terminally modified by the addition of the H4 or D4 Nic-NHS ester freshly prepared in pH 8.5, 50 mM sodium phosphate buffer. Further aliquots were added after 10 and 20 minutes. After 2 hours 0.5 M hydroxylamine in pH 8.5 sodium phosphate buffer was added and the solution left overnight. The reaction was stopped by the addition of 2 μl of formic acid.

MALDI Analysis and Quantification

Crude digest (0.5 μl) was desalted using a Ziptip™ (Millipore, Bedford, Mass., USA) and eluted with 70% methanol, 1% acetic acid and was co-crystallised with the same amount of matrix solution (10 mg/ml α-cyano-4-hydroxy-cinnamic acid in 50% acetonitrile, 1.25% TFA in water). The dry sample-matrix mix was washed three times with ice-cold 1% TFA to the mixture on the MALDI target. Mass spectra were recorded using a Voyager Elite MALDI-TOF mass spectrometer (Perseptive Biosystems, Framingham, Mass., USA) operated in delayed extraction reflector mode using an accelerating voltage of 20 kV, a pulse delay time of 150 ns, a grid voltage of 60% and a guide wire voltage of 0.05%. Spectra were accumulated for 32 laser shots. The masses obtained were used to search sequence databases (Swissprot release 38 and nrdb release Jan. 10, 1999) with the MassSearch and PeptideSearch programs (18,19). The ratio of the D4 and H4 labelled peptides were calculated from the relative peak heights and averaged over all peptides found to be unique to an identified protein.

MS/MS Analysis and De Novo Sequencing

MS/MS sequencing was performed on a Finnigan MAT LC-Q ion trap mass spectrometer (San Jose, Calif., USA). The desalted digest in 70% methanol and 1% acetic acid was loaded into a home-made nanospray tip and electrosprayed into the mass spectrometer at a flow rate of 0.2 μl/min using a syringe pump. The peaks of interest were selected with a mass window wide enough to include the entire isotope distribution. Fragmentation was carried out using a relative collision energy of 35-60 units for MH⁺ ions and 20-35 units for MH²⁺ ions. The spectra were manually interpreted and the deduced sequences were used for (T)FASTA homology searches (20).

### Results and Discussion

Experimental Principle

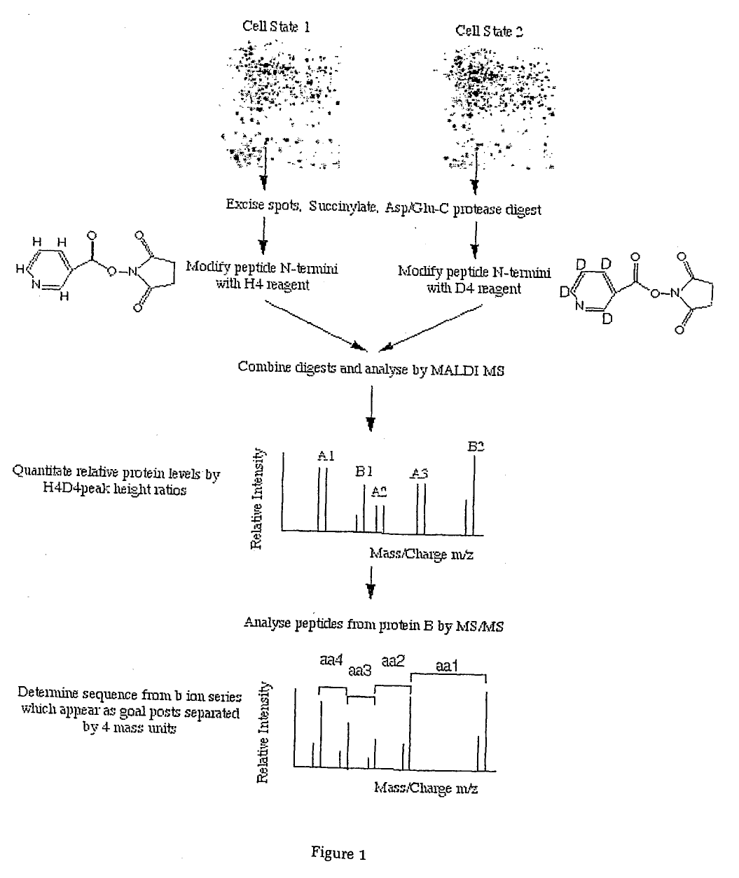

The isotopic labelling of proteins extracted from ½D PAGE gels allows the quantitation of the changes in protein expression occurring between two different biological states (FIG. 1). The proteins are first succinylated to block the epsilon amino groups of lysine before digestion. Each expression state is then uniquely identified by reacting the N-terminus of the corresponding peptide products with a light or heavy isotopic label. Quantitation is carried out by combining the digests and the ratio of the amount of protein present in each state can then be obtained from the ratio of the light and heavy labels. These ratios are deduced from the peak heights of the peaks obtained in the MALDI mass spectrum, which are separated by four mass units. Even if multiple proteins are present in a spot, the individual expression ratios for each protein can be obtained. In FIG. 1, the expression of protein A remains constant in the two states whereas that of B is increased by a factor of three. This isotopic labelling pattern simultaneously facilitates de novo peptide sequencing by mass spectrometry. The fixation of a positive charge at the N-terminal strongly favours the formation of b-ions (those which contain the N-terminus) during MS/MS fragmentation. These can be readily identified since they appear as a doublet separated by four mass units in contrast to the y ions (those arising from the C-terminal) which appear as a singlet. Thus the deduction of the peptide sequence from the fragmentation spectrum is greatly facilitated.

De Novo Peptide Sequencing

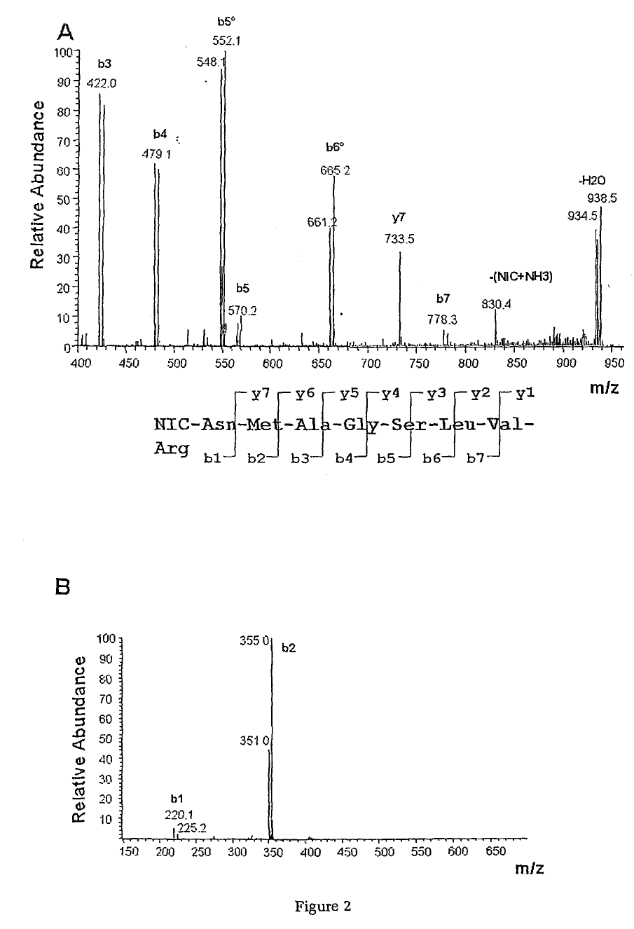

The protein is first succinylated on the lysine side chains which allows a selective nicotinylation at the N-termini to be achieved quantitatively. The only side reaction seen is the succinlyation or nicotinylation of tyrosine which can be reversed by hydroxylamine treatment. The MS/MS spectrum of a peptide (m/z 1+952/956) from an Escherichia coli protein in shown in FIG. 2A. The b-ion series can be easily identified and provides complete coverage despite the presence of a C-terminal arginine that strongly favours the formation y-ions. The limited MS/MS mass range of the ion trap (only ions down to ca. ⅔ of the parent mass can be effectively trapped) usually precludes the accumulation of a complete ion series. In this case, the dominant b3 ion (422/426) can easily be identified, isolated and fragmented in an MS³ experiment providing complete sequence coverage to the N-terminal (FIG. 2B). The complete sequence (NMAGSLVR) can be rapidly extracted. The sequence was used in a FASTA homology search to identify the protein as the E. coli 50S ribosomal subunit protein L17. In a short twenty-minute data accumulation, peptide sequences were obtained for 47 residues from 127 possible, giving a 37% coverage.

The placement of the highly basic nicotinyl group on the N-terminal of all the peptides has several advantages over C-terminal labelling using ¹⁸O (¹²). The yield of b-ions is greatly increased and these are easily identified by their isotopic pattern (goalposts separated by four mass units). The method can be applied to any proteolytic or chemical digestion and it is far less expensive and more reproducible that the use of ¹⁸O labelled water (¹³). The increase in b-ion intensity and the ease of recognition of these ions as doublets makes it possible to obtain full-length sequence coverage of peptides of m/z>1,000 in an ion trap, extending the usual sequencing range. Finally, protein succinylation increases the yield of b ions by suppressing charge localisation by internal lysines and also allows one to differentiate between lysine and glutamine in low energy MS/MS fragmentation regimes.

Isotopic Quantitation of Proteins Separated by 2D Electrophoresis

E. coli has served as a model system for 2D-gel analysis of comprehensive global protein expression since the technique was first developed (14). Despite the 4,288 protein-encoding genes that have been annotated in the genome, only 1600 spots have been visualised on 2D gels to date (15). One of the main reasons is that often several proteins co-migrate in a single spot. In order to deal with this problem, the present inventors have applied the isotope labelling approach (FIG. 1) according to the present invention to allow quantitation of proteins obtained from bands/spots from 1 and 2D gels.

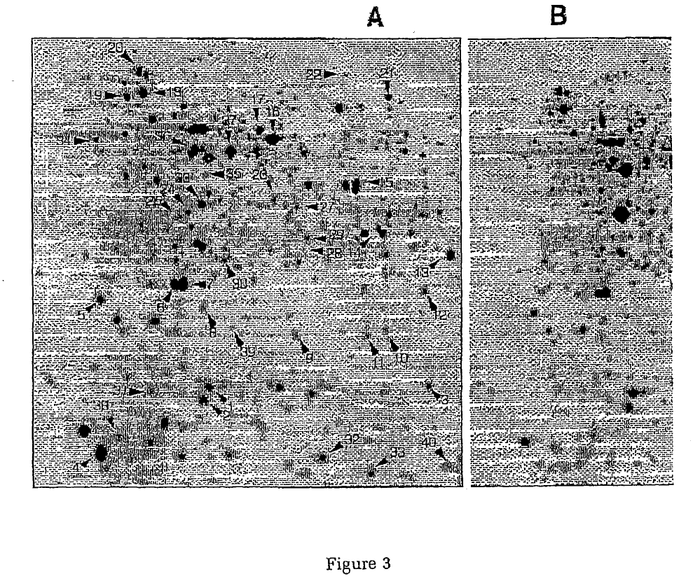

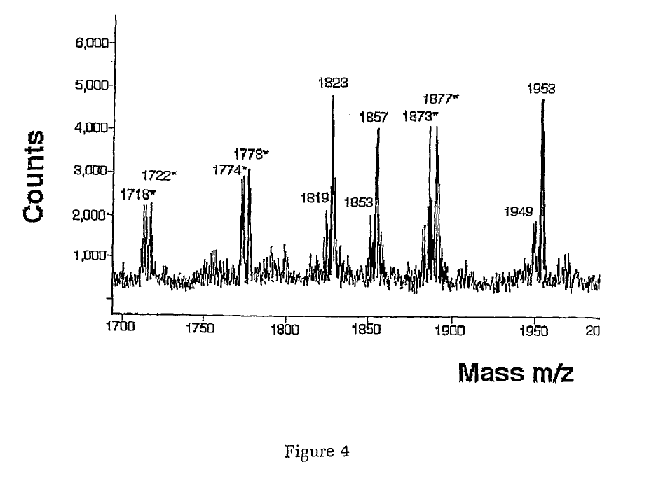

The effect of carbon source restriction on the protein expression profile of E. coli was visualised by 2D PAGE (FIG. 3). Forty high and low intensity spots were excised, succinylated and labelled with light reagent for the unlimited and heavy reagent for restricted carbon growth. The ratio of isotopes were determined for all the spots and compared with the integrated optical density ratios obtained from the gel scans (Table 1). The isotopic ratios showed the same variability (standard deviations of around +/−0.05) as the optical density values and roughly the same absolute values, but had three main advantages. Firstly, the isotopic values were unaffected by saturation which frequently happens with staining methods (in this case spots 4, 6, 7, 16, 18, 23, 36 and 37). Secondly, ratios could be obtained for extremely weak staining spots that either could not be determined using the 2D-analysis software (spots 28, 31, 32, and 39). Thirdly, the most important advantage of the isotopic method was the ability to quantitate multiple proteins occurring in a single spot. Spot 37 (FIG. 3) was too dense in both gels to allow analysis by optical density however by eye a clear decrease could be seen. MALDI MS analysis of the labelled digests showed clearly that two proteins were present, one, recA whose expression level remained unchanged, and a second, malE, whose level was increasing by a factor of three (FIG. 4) under starvation conditions. The amount of the two proteins relative to one another is not possible to judge due to the differences in ionisation efficiency of peptides with different sequences. Determining the relative amounts of a protein in state 1 and 2 is possible since the sequences are identical and the peptides differ only in the hydrogen/deuterium substitution of the tag and at least 10 peptides are used to calculate the change in protein expression. If a spot is completely de novo induced, it can be modified with a 50:50 mixture of D4H4 NicNHS to enable rapid sequencing.

The method for quantitation and de novo sequencing according to the present invention is universally applicable and is does not require a protein to contain a certain amino acid such as cysteine or lysine. Moreover, the method does not require in vivo metabolic labelling. The method is ideally suited for the analysis of changes in membrane protein expression since these proteins can be partially separated by 1D SDS-PAGE and the isotope labelling allows one to quantitate multiple proteins in a single band. The method is also open to automation and allows flexible sequence searching in databases, allowing for differences due to homologies and post-translational modifications.

TABLE 1. A comparison of protein quantitation by optical density and isotope ratios. The proteins spots labelled in FIG. 4 were succinylated, digested with Asp/GluC protease and then modified with the light or heavy reagent as outlined in FIG. 1. The D4/H4 ratios obtained from the MS spectrum were calculated from the relative peaks heights in the MALDI spectrum. These are compared with optical density ratios obtained from scanning the Coomassie stained gels in a laser densitometer. For spots containing more than one protein, only the ratio of the protein changing is taken. Intensely staining spots which show saturation are indicated by * and those too weak to be quantitated by the 2D analysis software are marked by #.

| Spot number | OD1/OD2: ratio | Isotope ratio |
|---|---|---|
| 1 | 0.47 | 0.55 |
| 2 | 0.78 | 0.85 |
| 3 | 1.38 | 1.45 |
| *4 | 4.7 | 25.1 |
| 5 | 2.71 | 3.21 |
| *6 | 1.4 | 2.55 |
| *7 | 1.7 | 3.18 |
| 8 | 5.11 | 6.21 |
| 9 | 1.67 | 1.77 |
| 10 | 6.58 | 7.58 |
| 11 | 3.03 | 3.13 |
| 12 | 1.64 | 1.94 |
| 13 | 1.85 | 1.93 |
| 14 | 2.44 | 2.38 |
| 15 | 0.89 | 0.90 |
| *16 | 1.87 | 1.11 |
| 17 | 1.0 | 1.13 |
| *18 | 0.95 | 1.42 |
| 19 | 2.89 | 3.12 |
| 20 | 0.63 | 0.74 |
| 21 | 1.73 | 1.54 |
| 22 | 0.88 | 0.81 |
| *23 | 0.21 | 0.07 |
| 24 | 1.88 | 1.65 |
| 25 | 2.40 | 2.28 |
| 26 | 2.08 | 2.13 |
| 27 | 1.29 | 1.22 |
| #28 | 0.22 | 0.002 |
| 29 | 0.45 | 0.39 |
| 30 | 0.91 | 0.76 |
| #31 | 5.22 | 22.1 |
| #32 | 12.3 | 89.0 |
| 33 | 0.75 | 0.79 |
| 34 | 3.02 | 2.91 |
| 35 | 0.28 | 0.35 |
| *36 | 0.78 | 1.11 |
| *37 | 0.92 | 0.35 |
| 38 | 1.61 | 1.77 |
| #39 | 1.12 | 2.55 |
| 40 | 0.72 | 1.13 |

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

REFERENCES

1. Oda, Y., Huang, K., Cross, F. R., Cowburn, D. & Chait, B. T. Accurate quantitation of protein expression and site-specific phosphorylation. Proc Natl Acad Sci USA 96, 6591-6596 (1999).

2. McConkey, E. H. Double-label autoradiography for comparison of complex protein mixtures after gel electrophoresis. Anal Biochem 96, 39-44 (1979).

3. Cottrell, J. S. Protein identification by peptide mass fingerprinting. Pept Res 7, 115-124 (1994).

4. Eng, J. K., McCormack, A. L. & Yates, J. R. Correlating tandem mass spectral data of peptides to sequences in a protein database. J Am Soc Mass Spec 5, 976-989 (1994).

5. Mann, M. & Wilm, M. Error-tolerant identification of peptides in sequence databases by peptide sequence tags. Anal Chem 66, 4390-4399 (1994).

6. Hunt, D. F., Yates, J. R. d., Shabanowitz, J., Winston, S. & Hauer, C. R. Protein sequencing by tandem mass spectrometry. Proc Natl Acad Sci U S A 83, 6233-6237 (1986).

7. Johnson, R. S. & Biemann, K. Computer program (SEQPEP) to aid in the interpretation of high-energy collision tandem mass spectra of peptides. Biomed Environ Mass Spectrom 18, 945-957 (1989).

8. Hunt, D. F., Buko, A. M., Ballard, J. M., Shabanowitz, J. & Giordani, A. B. Sequence analysis of polypeptides by collision activated dissociation on a triple quadrupole mass spectrometer. Biomed Mass Spectrom 8, 397-408 (1981).

9. Spengler, B. et al. Peptide sequencing of charged derivatives by post-source decay MALDI mass spectrometry. Int. J. Mass Spectrom. 169/170, 127-140 (1997).

10. Stults, J. T., Lai, J., McCune, S. & Wetzel, R. Simplification of high-energy collision spectra of peptides by amino-terminal derivatization. Anal Chem 65, 1703-1708 (1993).

11. Gygi, S. P. et al. Quantitative analysis of complex protein mixtures using isotope-coded affinity tags. Nat Biotechnol 17, 994-999 (1999).

12. Takao, T. et al. Facile assignment of sequence ions of a peptide labelled with 18O at the carboxyl terminus. Rapid Commun Mass Spectrom 5, 312-315 (1991).

13. Schnolzer, M., Jedrzejewski, P. & Lehmann, W. D. Protease-catalyzed incorporation of 18O into peptide fragments and its application for protein sequencing by electrospray and matrix-assisted laser desorption/ionization mass spectrometry. Electrophoresis 17, 945-953 (1996).

14. Pederson, S., Bloch, P. L., Reeh, S. & Neidhart, F. C. Patterns of protein synthesis in E. coli: a catalog of the amount of 140 individual proteins at different growth rates. Cell 14, 179-190 (1978).

15. VanBogelen, R. A., Abshire, K. Z., Moldover, B., Olson, E. R. & Neidhardt, F. C. Escherichia coli proteome analysis using the gene-protein database. Electrophoresis 18, 1243-1251 (1997).

16. Kertesz, M. A., Leisinger, T. & Cook, A. M. Proteins induced by sulfate limitation in Escherichia coli, Pseudomonas putida, or Staphylococcus aureus. J Bacteriol 175, 1187-1190 (1993).

17. Dainese, P. et al. Probing protein function using a combination of gene knockout and proteome analysis by mass spectrometry. Electrophoresis 18, 432-442 (1997).

18. James, P., Quadroni, M., Carafoli, E. & Gonnet, G. Protein identification by mass profile fingerprinting. Biochem Biophys Res Commun 195, 58-64 (1993).

19. Mann, M., Hojrup, P. & Roepstorff, P. Use of mass spectrometric molecular weight information to identify proteins in sequence databases. Biol Mass Spectrom 22, 338-345 (1993).

20. Pearson, W. R. Rapid and sensitive sequence comparison with FASTP and FASTA. Methods Enzymol 183, 63-98 (1990).

1. A method of labelling a protein, which method comprises the steps of:
(a) treating the protein with a protecting agent so as to block epsilon amino acid groups present on lysines residues of the protein;
(b) cleaving the protein into a peptide mixture; and
(c) treating the peptide mixture with a labelled agent which binds to N-terminal amino acids of the peptides.

2. A method according to claim 1 wherein step (a) is succinylation of said protein.

3. A method according to claim 2 wherein the protecting agent is succinic anhydride.

4. A method according to any one of the preceding claims wherein the cleaving in step (b) is by incubation with Asp/GluC (V8) protease.

5. A method according to any one of the preceding claims wherein step (c) is treating with 1-(H4/D4 nicotinoyloxy) succinimide (H4 or D4 Nic-NHS) ester.

6. A method of identifying and/or characterising a protein which method comprises labelling a protein by the method of any one of claims 1 to 5 followed by the step of:
(d) detecting or measuring the amount of label on the peptides.

7. A method according to claim 6 wherein the detection or measuring is by mass spectroscopy.

8. A method according to claim 7 wherein said mass spectroscopy is MALDI mass spectroscopy.

9. A method according to any one of claims 6 to 8 which further comprises the step of:
(e) identifying the amino acid sequence of the labelled peptides.

10. A method according to claim 9 wherein step (e) is by mass spectral analysis using an ion trap mass spectrometer.

11. A method according to any one of the preceding claims wherein the protein has been separated from other proteins prior to labelling.

12. A method according to claim 11 wherein the protein has been separated by 1D or 2D polyacrylamide gel electrophoresis (PAGE).

13. A method of comparing or determining the expression of a protein in a first cell and a second cell, the method comprising the steps of:
(i) obtaining the protein from a first cell and a second cell;
(ii) labelling the protein from the first cell and the second cell by the method of any one of claims 1 to 5 wherein a first labelling agent is used to label the the protein from the first cell and a second labelling agent is used to label the the protein from the second cell and wherein the first and second labelling agent can be distinguished; and
(iii) detecting or measuring the amount of first and second labelling agent on the peptides.

14. A method according to claim 13 wherein the first and second labelling agent can be distinguished on the basis of mass.

15. A method according to claim 14 wherein step (iii) is MALDI MS.

16. A method according to claim 14 or claim 15 wherein the first labelling agent comprises a light isotopic label and the second labelling agent comprises a heavy isotopic label, or vice versa.

17. A method according to claim 16 wherein the first and second labelling agents are 1-(H4 nicotinoyloxy)succinimide (H4 Nic-NHS) ester and 1-(D4 nicotinoyloxy) succinimide (D4 Nic-NHS) ester.

18. A method according to any one of claims 13 to 17 which further comprises the step of:
(iv) identifying the amino acid sequence of the labelled peptides.

19. A method according to claim 18 wherein step (iv) is by mass spectral analysis using an ion trap mass spectrometer.

20. A method according to any one of claims 13 to 19 wherein the labelled peptide mixtures are combined prior to step (iii).

21. Use of two or more differentially isotopically-labelled succinylating agents as labelling agents for peptides and/or proteins.

22. Use according to claim 21 wherein said agents are 1-(H4 nicotinoyloxy) succinimide and 1-(D4 nicotinoyloxy) succinimide esters.
